(19) United States
(12) Patent Application Publication
(10) Pub. No.: US 2019/0150481 A1
(43) Pub. Date: May 23, 2019

(54) COMPOSITIONS AND METHODS OF IMPROVING DIETARY PHOSPHORUS AND CALCIUM UTILIZATION IN ANIMALS

(71) Applicant: Novus International, Inc., St. Charles, MO (US)

(72) Inventors: Ping Ren, St. Charles, MO (US); Karen Wedekind, St. Charles, MO (US); Mercedes Vazquez-Anon, St. Charles, MO (US)

(21) Appl. No.: 16/190,385

(22) Filed: Nov. 14, 2018

Related U.S. Application Data
(60) Provisional application No. 62/587,708, filed on Nov. 17, 2017.

(51) Int. Cl. A23K 20/189; A23K 20/105; A23K 20/24; A23K 20/22

(52) U.S. Cl. CPC A23K 20/189; A23K 20/105; C07C 323/22; A23K 20/22; C12Y 301/03008; A23K 20/24

(57) ABSTRACT

The present invention relates to compositions and methods for improving dietary phosphorus and calcium utilization in animals.

### CROSS-REFERENCE TO RELATED APPLICATIONS

This application claims the benefit of U.S. Provisional Application No. 62/587,708, filed November 17, 2017, which is hereby incorporated by reference in its entirety.

### FIELD

The present disclosure relates to compositions and methods for improving dietary phosphorus and calcium utilization in animals.

### BACKGROUND

Following protein and energy sources, phosphorus (P) is the next most expensive nutrient in the production of animals, such as poultry, swine, and ruminants. Various sources of phosphorus are fed to animals to maximize growth and performance. However, a significant amount of the phosphorus fed to animals is excreted into the feces and urine. Further, the excreted phosphorus is known to pollute local water sources.

Since feed is a major cost in animal production, it is desirable to supplement their rations with compounds or compositions that allow the animal to digest more of the nutrients in the feed. Traditionally, animal feed is supplemented with phytases to extract or hydrolyze the phosphorus from the phytate molecule present in the feed. However, phytases are not always able to extract all of the phosphorus from the phytate molecule. Consequently, there is a need for compositions and methods that increase the utilization of dietary phosphorus.

### SUMMARY

One aspect of the present disclosure encompasses a composition comprising a metal chelate and a phytase, wherein the composition comprises about 50 ppm to about 1800 ppm contains of the metal chelate and about 6 ppm to about 1000 ppm of the phytase, and the metal chelate comprises at least one metal ion and at least one ligand of Formula (III):

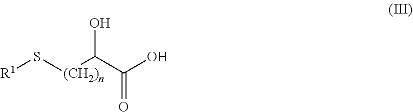

wherein:

n is an integer from 1 to 5; and

R¹ is C₁ to C₆ alkyl or C₁ to C₆ substituted alkyl.

An additional aspect of the present disclosure encompasses a method for improving dietary phosphorus and calcium utilization in an animal. The method comprises administering to the animal a metal chelate and a phytase, wherein dietary phosphorus and calcium utilization is increased relative to administration of an inorganic salt of the metal and the phytase.

Another aspect of the present disclosure encompasses a method for improving dietary phosphorus and calcium utilization in an animal fed a diet comprising a phytase. The method comprises administering to the animal a metal chelate rather than an inorganic salt of the metal.

Other aspects and features of the invention will be in part apparent and in part pointed our hereinafter.

### DETAILED DESCRIPTION

The addition of metal chelates to phytases has been discovered to increase the effectiveness of the phytases to hydrolyze phosphorus and calcium from complex compounds, e.g., phytate. Without being bound by theory, it is believed that the metal chelates prevent dietary antagonism of phytase. Further, it is believed, that trace minerals reduce the effectiveness of phytases in animal feed. It has also been discovered, as illustrated in the examples, that the metal chelate and phytase composition of the present disclosure when added to or supplemented to an animal's feed increases digestibility and utilization of calcium and phosphorus and as a result reduced the amount of phosphorus being released into the environment.
(I) Compositions

One aspect of the present disclosure encompasses a metal chelate and a phytase. Each of the components of the composition is detailed below.

(a) Metal Chelate

The composition comprises a metal chelate of Formula (I):

LₓM^{Y} (I)

wherein

L is a ligand,

M is a metal ion, and

x and y are integers from 1 to 10.

(i) Metal Ions

The metal ion may be, but not limited to, calcium, chromium, cobalt, copper, germanium, iron, lithium, magnesium, manganese, molybdenum, nickel, potassium, sodium, rubidium, tin, vanadium, and zinc. In a preferred embodiment, the metal may be selected from the group consisting of calcium, magnesium, zinc, iron, copper, manganese, sodium, potassium, cobalt, and nickel. In a more preferred embodiment, the metal cation may be selected from the group consisting of zinc, iron, copper, and manganese. In an exemplary embodiment, the metal may be zinc. In another exemplary embodiment, the metal may be copper. In an additional exemplary embodiment, the metal may be manganese.

In general, x may be an integer from 1 to 10. In an embodiment, x may be an integer from 1 to 5 or from 1 to 3. In some embodiments, x may be 1, 2, 3, 4, 5, 6, 7, 8, 9, or 10. In a preferred embodiment, x may be 2.

In an embodiment, y is the oxidation state of the metal ion. In general, y may be an integer from 1 to 10. In an embodiment, y may be an integer from 1 to 5 or from 1 to 3. In some embodiments, y may be 1, 2, 3, 4, 5, 6, 7, 8, 9, or 10. In a preferred embodiment, y may be 2. In an exemplary embodiment, the metal ion may be divalent zinc. In another exemplary embodiment, the metal ion may be divalent copper. In still another exemplary embodiment, the metal ion may be divalent manganese.

In an embodiment, x and y may be integers form 1 to 3. In an exemplary embodiment, x and y may be 2.

In general, the amount of metal ion in the compositions may range from about 1 ppm to about 300 ppm. In some embodiments, the amount of metal ion in the composition may range from about 1 ppm to about 300 ppm. In a further embodiment, the amount of metal ion in the composition may be about 1, about 5, about 10, about 25, about 50, about 75, about 100, about 125, about 150, about 175, about 200, about 225, about 250, about 275, or about 300 ppm. In an exemplary embodiment, the amount of metal ion in the composition may range from about 10 to about 200 ppm.

(ii) Ligand

The ligand of the metal chelate may be, but not limited to, organic acid moieties, amino acid moieties, or derivatives thereof.

In some embodiments, the ligand may be an organic acid moiety. Suitable organic acid moieties may be, but not limited, to adipate, ascorbate, caprylate, citrate, furmarate, glucoheptonate, gluconate, glutarate, glycerophosphate, lactate, ketoglutarate, malate, malonate, orotate, oxlate, pantothenate, picolinate, pidolate, sebacate, succinate, and tartrate.

In other embodiments, the ligand may be an amino acid moiety. Suitable amino acid derivatives may be, but not limited to, alanate, arginate, asparaginate, aspartate, cysteinate, glutaminate, glutamate, histidinate, homocysteinate, isoleucinate, lysinate, methionate, phenylalinate, prolinate, serinate, threonate, typtophanate, tyrosinate, and valinate.

In exemplary embodiments, the ligand may be methionine or a hydroxy analog of methionine, wherein the ligand is a compound of Formula (II):

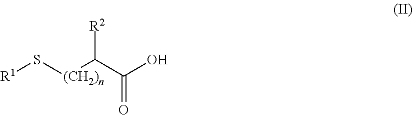

wherein:

R¹ is alkyl or substituted alkyl;

R² is hydroxyl or amino; and

n is an integer from 1 to 5.

In some embodiments, R¹ may be C₁ to C₆ alkyl or C₁ to C₆ substituted alkyl. In further embodiments, R¹ may be methyl, ethyl, propyl, isopropyl, butyl, tert-butyl, hexyl, cyclohexyl, and the like. In specific embodiments, R¹ may be methyl. In certain embodiments, n may be 1, 2, 3, 4, or 5. In specific embodiments, n may be 1 or 2.

In one embodiment, R¹ may be methyl, R² may be amino, and n may be 1 or 2. In a specific embodiment, R¹ may be methyl, R² may be hydroxyl, and may be 2.

In another exemplary embodiment, the ligand may be a hydroxy analog of methionine, wherein the ligand is a compound of Formula (III):

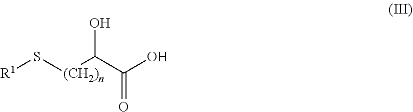

wherein:

n is an integer from 1 to 5; and

R¹ is C₁ to C₆ alkyl or C₁ to C₆ substituted alkyl.

In some embodiments, n may be 1, 2, 3, 4, or 5. In specific embodiments, n may be 1 or 2. In some embodiments, R¹ may be C₁ to C₆ alkyl or C₁ to C₆ substituted alkyl. In further embodiments, R¹ may be methyl, ethyl, propyl, isopropyl, butyl, tert-butyl, hexyl, cyclohexyl, and the like. In specific embodiments, R¹ may be methyl. The compound formed by this selection of chemical groups is 2-hydroxy-4-(methylthio)butanoic acid (commonly known as “HMTBA” and sold by Novus International, St. Louis, Mo. under the trade name ALIMET). HMTBA, as used herein, included monomers, dimers, trimers, and longer oligomers that include a greater number of repeating units.

(iii) Chelate

Typically, one or more ligands complex with one or more metal ions to form the chelate of Formula (I). Irrespective of the embodiment, suitable non-limiting examples of metal ions are described in Section (I)(a)(i).

Generally speaking, a suitable ratio of ligand to metal ion is from about 1:1 to about 3:1 or higher. In another embodiment, the ratio of ligand to metal ion is from about 1.5:1 to about 2.5:1. Of course within a given mixture of metal chelate compounds, the mixture will include compounds having different ratios of ligand to metal ion. For example, a composition of metal chelate compounds may have species with ratios of ligand to metal ion that include 1:1, 1.5:1, 2:1, 2.5:1, and 3:1.

In embodiments in which the ligand is a compound of Formula (II), the chelate comprises one or more ligands having Formula (II) together with one or more metal ions. In each embodiment, the ligand compound having Formula (II) is preferably HMTBA. In one exemplary embodiment, the metal chelate is Mn(HMTBA)₂. In a further exemplary embodiment, the metal chelate is Cu(HMTBA)₂. In an alternative exemplary embodiment, the metal chelate is Zn(HMTBA)₂.

As will be appreciated by a skilled artisan, the ratio of ligands to metal ions forming a metal chelate compound of Formula (I) can and will vary. Generally speaking, where the number of ligands is equal to the charge of the metal ions, the charge of the molecule is typically net neutral because the carboxyl moieties of the ligands having Formula (II) are in deprotonated form. By way of further example, in a chelate species where the metal ion carries a charge of 2⁺and the ligand to metal ion ratio is 2:1, each of the hydroxyl or amino groups (i.e., R² of compound II) is believed to be bound by a coordinate covalent bond to the metal while an ionic bond exists between each of the carboxylate groups of the metal ion. This situation exists, for example, where divalent zinc, copper, or manganese is complexed with two HMTBA ligands. By way of further example, where the number of ligands exceeds the charge on the metal ion, such as in a 3:1 chelate of a divalent metal ion, the ligands in excess of the charge generally remain in a protonated state to balance the charge. Conversely, where the positive charge on the metal ion exceeds the number of ligands, the charge may be balanced by the presence of another anion, such as, for example, chloride, bromide, iodide, bicarbonate, hydrogen sulfate, and dihydrogen phosphate.

Metal chelate compounds of the invention may be made in accordance with methods generally known in the art, such as described in U.S. Pat. Nos. 4,335,257 and 4,579,962, which are both hereby incorporated by reference in their entirety. Alternatively, the metal chelate compounds may be purchased from a commercially available source. For example, Zn(HMTBA)₂ and Cu(HMTBA)₂ may be purchased from Novus International, Saint Louis, Mo., sold under the trade names MINTREX Zn, and MINTREX Cu, respectively.

In general the amount of metal chelate in the composition may range from about 30 to about 1800 ppm. In further embodiments, the amount of metal chelate in the composition may be about 30, about 40, about 50, about 75, about 100, about 150, about 200, about 250, about 300, about 350, about 400, about 450, about 500, about 550, about 600, about 650, about 700, about 750, about 800, about 850, about 900, about 950, about 1000, about 1100, about 1150, about 1200, about 1250, about 1300, about 1350, about 1400, about 1450, about 1500, about 1550, about 1600, about 1650, about 1700, about 1750, or about 1800 ppm. In an exemplary embodiment, the amount of metal chelate in the composition may range from about 60 to about 1600 ppm.

(b) Phytase

The composition also comprises a phytase. Phytases are enzymes that catalyze the hydrolysis of O—P bonds in phytate in grains and oil seeds and release digestible inorganic phosphorous (and calcium or other divalent metal cation complexed with phytate).

The phytase enzyme may be derived from fungi, yeast, bacteria, protozoa, or plants, wherein the fungi or bacteria may be thermophilic. The phytase maybe an acid phytase, an alkaline phytase, a 3-phytase, or a 6-phytase. The phytase may be a wild type phytase or a modified or variant phytase comprising at least one amino acid substitution. Modified or variant phytases may have improved biochemical properties, such as improved pH activity, improved pH stability, improved thermostability, improved specific activity, improved kinetics, improved stability to proteases, and the like. The phytase may be isolated from the original organism or the phytase may be recombinantly produced (i.e., expressed in yeast or another system).

In some embodiments, the phytase may be a fungal phytase derived from Aspergillus niger, Aspergillus oryzae, Aspergillus fecuum, Aspergillus awamori, Aspergillus nidulans, Aspergillus fumigatus, Aspergillus terreus, Peniophora lycii, Cladosporium sp., Myceliophtora thermophila, Talaromyces thermophilus, Thermomyces lanuginosus, or Mucor pusillus. In other embodiments, the phytase may be a yeast phytase derived from Saccharomyces spp., such as Saccharomyces cerevisiae, Kluyveromyces spp., such as Kluyveromyces lactis, Arxula adeninivorans, Candida Krusei, Pichia anomala, or Schwanniomyces castillii. In still other embodiments, the phytase may be a bacterial phytase derived from Bacillus sp., such as Bacillus subtilis, Pseudomonas sp., such as Pseudomonas syringae, Escherichia coli, Selenomonas sp., Mitsuokella multiacidus, Citrobacter braaki, Obesumbacterium proteus, Klebsiella spp., or Shewanella oneidensis. In additional embodiments, the phytase may be a protozoan phytase derived from Paramecium tetraurelia. In further embodiments, the phytase may be a plant phytase derived from Avena sativa (oats), Hordeum vulgare (barley), Oryza sativa (rice), Secale cereale (rye), sorghum bicolor, Triticum aestivum, Triticum durum, Triticum spelta (wheat species), Glycine max (soybean), Zea mays (corn), or Lilium spp. (lilies). In specific embodiments, the phytase may be of fungal, yeast, or bacterial origin.

In general, the amount of phytase in the composition may range from about 6 ppm to about 1000 ppm. In certain embodiments, the amount of phytase in the composition may be about 6, about 8, about 10, about 25, about 50, about 100, about 150, about 200, about 250, about 300, about 350, about 400, about 450, about 500, about 550, about 600, about 650, about 700, about 750, about 800, about 850, about 900, about 950, or about 10000 ppm. In specific embodiments, the amount of phytase in the composition may range from about 8 ppm to about 800 ppm.

Phytase activity is expressed in phytase units (FTU). One phytase unit (FTU) is defined as the amount of enzyme that liberates 1 micromole of inorganic phosphorus per minute from 0.0051 mol/l sodium phytate at 37° and pH 5.50 under the conditions of the test. In an embodiment, the amount of phytase in the composition may range from about 2000 to about 12000 FTU per g of composition. In a further embodiment, the amount of phytase in the composition may be about 2000, about 2250, about 2500, about 2750, about 3000, about 3250, about 3500, about 3750, about 4000, about 4250, about 4500, about 4750, about 5000, about 5250, about 5500, about 5750, about 6000, about 6250, about 6500, about 6750, about 7000, about 7250, about 7500, about 7750, about 8000, about 8250, about 8500, about 8750, about 9000, about 9250, about 9500, about 9750, about 10000, about 10250, about 10500, about 10750, about 11000, about 11250, about 11500, about 11750, or about 12000. In an exemplary embodiment, the amount of phytase in the composition may range from about 4000 to about 10000 FTU per g of composition.

(c) Excipients

The compositions may comprise a variety of excipients. Suitable excipients include fillers, binders, pH regulating agents, disintegrants, dispersing agents, preservatives, lubricants, coloring agents, flavoring agents, taste masking agents, or combinations thereof. In general, the excipient is a grade suitable for use in a nutritional composition.

In some embodiments, the excipient may comprise at least one filler. Non-limiting examples of suitable fillers (also called diluents) include cellulose, microcrystalline cellulose, cellulose ethers (e.g., ethyl cellulose, methyl cellulose, hydroxypropyl cellulose, hydroxypropyl methylcellulose, sodium carboxymethylcellulose, etc.), cellulose esters (i.e., cellulose acetate, cellulose butyrate, and mixtures thereof), starches (e.g., corn starch, rice starch, potato starch, tapioca starch, and the like), modified starches, pregelatinized starches, phosphated starches, starch-lactose, starch-calcium carbonate, sodium starch glycolate, glucose, fructose, sucrose, lactose, xylose, lactitol, mannitol, malitol, sorbitol, xylitol, maltodextrin, trehalose, calcium carbonate, calcium sulfate, calcium phosphate, calcium silicate, magnesium carbonate, magnesium oxide, talc, or combinations thereof.

In other embodiments, the excipient may comprise at least one binder. Examples of suitable binders include, without limit, starches (e.g., corn starch, potato starch, wheat starch, rice starch, and the like), pregelatinized starch, hydrolyzed starch, cellulose, microcrystalline cellulose, cellulose derivatives (e.g., methylcellulose, ethylcellulose, hydroxypropylcellulose, hydroxypropylmethylcellulose, sodium carboxymethylcellulose, and the like), saccharides (e.g., sucrose, lactose, and so forth), sugar alcohols (e.g., maltitol, sorbitol, xylitol, polyethylene glycol, and the like), alginates (e.g., alginic acid, alginate, sodium alginate, and so forth), gums (e.g., gum arabic, guar gum, gellan gum, xanthan gum, and the like), pectins, gelatin, C₁₂-C₁₈ fatty acid alcohols, polyvinylpyrrolidinone (also called copovidone), polyethylene oxide, polyethylene glycol, polyvinyl alcohols, waxes (e.g., candelilla wax, carnauba wax, beeswax, and so forth), or combinations of any of the foregoing.

In still other embodiments, the excipient may be a pH regulating agent. By way of non-limiting example, pH regulating agents include organic carboxylic acids (e.g., acetic acid, ascorbic acid, citric acid, formic acid, glycolic acid, gluconic acid, lactic acid, malic acid, maleic acid, propionic acid, succinic acid, tartaric acid, etc.) or salts thereof other acids (e.g., hydrochloric acid, boric acid, nitric acid, phosphoric acid, sulfuric acid, etc.), alkali metal or ammonium carbonates, bicarbonates, hydroxides, phosphates, nitrates, and silicates; and organic bases (such as, for example, pyridine, triethylamine (i.e., monoethanol amine), diisopropylethylamine, N methylmorpholine, N,N dimethylaminopyridine).

In additional embodiments, the excipient may be a disintegrant. Examples of suitable disintegrants include, without limit, povidone, crospovidone, croscarmellose sodium, sodium carboxymethylcellulose, carboxymethylcellose calcium, sodium starch glycolate, cellulose, microcrystalline cellulose, methylcellulose, silicon dioxide (also called colloidal silicone dioxide), alginates (e.g., alginic acid, alginate, sodium alginate, and so forth), clays (e.g., bentonite), or combinations thereof.

In alternate embodiments, the excipient may be a dispersing agent. Suitable dispersing agents include, but are not limited to, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In yet additional embodiments, the excipient may be a preservative. Non limiting examples of suitable preservatives include antioxidants (such as, e.g., alpha-tocopherol, ascorbyl palmitate, butylated hydroxyanisole, butylated hydroxytoluene, citric acid, dihydroguaretic acid, potassium ascorbate, potassium sorbate, propylgallate, sodium bisulfate, sodium isoascorbate, sodium metabisulfate, sorbic acid, 4-chloro-2,6-ditertiarybutylphenol, and so forth), antimicrobials (such as, e.g., benzyl alcohol, cetylpryidine chloride, glycerine, parabens, propylene glycol, potassium sorbate, sodium benzoate, sorbic acid, sodium propionate, and the like), or combinations thereof.

In still other embodiments, the excipient may be a lubricant. Examples of suitable lubricants include metal stearate such as magnesium stearate, calcium stearate, zinc stearate, a polyethylene glycol, a poloxamer, colloidal silicon dioxide, glyceryl behenate, light mineral oil, hydrogenated vegetable oils, magnesium lauryl sulfate, magnesium trisilicate, polyoxyethylene monostearate, sodium stearoyl fumarate, sodium stearyl fumarate, sodium benzoate, sodium lauryl sulfate, stearic acid, sterotex, talc, or combinations thereof.

In yet other embodiments, the excipient may be a color additive. Suitable color additives include, but are not limited to, food, drug and cosmetic colors (FD&C), drug and cosmetic colors (D&C), or external drug and cosmetic colors (Ext. D&C). drug and cosmetic colors (D&C), or external drug and cosmetic colors (Ext. D&C). These colors or dyes, along with their corresponding lakes, and certain natural and derived colorants may be suitable for use in the compositions.

In alternate embodiments, the excipient may be a flavoring agent. Flavoring agents may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot). In still another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. In still another embodiment, the excipient may include a taste-masking agent.

In some embodiments, the excipient may be a taste masking agent. Suitable taste masking agents include cellulose hydroxypropyl ethers (HPC); low-substituted hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; or combinations thereof.

(d) Physical Formulation

The compositions may be formulated into powders, pellets, liquids, crumbles, mash, etc. (e) Exemplary Composition

An exemplary composition comprises from about 8 ppm to about 800 ppm of phytase and from about 60 ppm to about 1600 ppm of a metal chelate comprising 2-hydroxy-4-(methylthio)butanoic acid (HMTBA). In specific embodiments, the metal ion is zinc, copper, or manganese, and the metal chelate is Zn(HMTBA)₂, Cu(HMTBA)₂, or Mn(HMTBA)₂.
(II) Animal Feed Premixes or Supplements

An additional aspect of the present disclosure encompasses a feed premix or supplement, or an animal feed ration comprising the compositions defined in Section (I). The feed ration may be formulated to meet the nutritional requirements of a variety of animals.

(a) Feed Premixes or Supplements

Another aspect of the present disclosure comprises an animal feed premix or feed supplement comprising the compositions described in Section (I). Typically, the premix will be added to various formulations of Feed (see Section (II)(b)) to formulate an animal feed ration. As will be appreciated by the skilled artisan, the particular premix or supplement can and will vary depending upon the feed ration and animal that the feed ration will be fed to. Accordingly, the premix or supplement may comprise a composition described in Section (I) and at least one bioactive agent.

Examples of suitable bioactive agents include vitamins, minerals, amino acids or amino acid analogs, antioxidants, organic acids, poly unsaturated fatty acids, essential oils, enzymes, prebiotics, probiotics, herbs, pigments, approved antibiotics, or combinations thereof.

In some embodiments, the bioactive agents may be one or more vitamins. Suitable vitamins include vitamin A, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid), vitamin B12, vitamin C, vitamin D, vitamin E, vitamin K, other B-complex vitamins (e.g., choline, carnitine, adenine), or combinations thereof. The form of the vitamin may include salts of the vitamin, derivatives of the vitamin, compounds having the same or similar activity of a vitamin, and metabolites of a vitamin.

In further embodiments, the bioactive agent may be one or more amino acids. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard amino acids (e.g., L-DOPA, GABA, 2-aminobutyric acid, and the like), amino acid analogs, or combinations thereof.

In alternate embodiments, the bioactive agent may be one or more antioxidants. Suitable antioxidants include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline (ethoxyquin), ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, n-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′, 5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives thereof, vitamin Q10, wheat germ oil, zeaxanthin, or combinations thereof.

In still other embodiments, the bioactive agent may be one or more organic acids. The organic acid may be a carboxylic acid or a substituted carboxylic acid. The carboxylic acid may be a mono-, di-, or tri-carboxylic acid. In general, the carboxylic acid may contain from about one to about twenty-two carbon atoms. Suitable organic acids, by way of non-limiting example, include acetic acid, adipic acid, butanoic acid, benzoic acid, cinnamaldehyde, citric acid, formic acid, fumaric acid, glutaric acid, glycolic acid, lactic acid, malic acid, mandelic acid, propionic acid, sorbic acid, succinic acid, tartaric acid, or combinations thereof. Salts of organic acids comprising carboxylic acids are also suitable for certain embodiments. Representative suitable salts include the ammonium, magnesium, calcium, lithium, sodium, potassium, selenium, iron, copper, and zinc salts of organic acids.

In yet other embodiments, the bioactive agent may be one or more poly unsaturated fatty acids. Suitable poly unsaturated fatty acids (PUFAs) include long chain fatty acids with at least 18 carbon atoms and at least two carbon-carbon double bonds, generally in the cis-configuration. In specific embodiments, the PUFA may be an omega fatty acid. The PUFA may be an omega-3 fatty acid in which the first double bond occurs in the third carbon-carbon bond from the methyl end of the carbon chain (i.e., opposite the carboxyl acid group). Suitable examples of omega-3 fatty acids include all-cis 7,10,13-hexadecatrienoic acid; all-cis-9,12,15-octadecatrienoic acid (alpha-linolenic acid, ALA); all-cis-6,9,12,15,-octadecatetraenoic acid (stearidonic acid); all-cis-8,11,14,17-eicosatetraenoic acid (eicosatetraenoic acid); all-cis-5,8,11,14,17-eicosapentaenoic acid (eicosapentaenoic acid, EPA); all-cis-7,10,13,16,19-docosapentaenoic acid (clupanodonic acid, DPA); all-cis-4,7,10,13,16,19-docosahexaenoic acid (docosahexaenoic acid, DHA); all-cis-4,7,10,13,16,19-docosahexaenoic acid; and all-cis-6,9,12,15,18,21-tetracosenoic acid (nisinic acid). In an alternative embodiment, the PUFA may be an omega-6 fatty acid in which the first double bond occurs in the sixth carbon-carbon bond from the methyl end of the carbon chain. Examples of omega-6 fatty acids include all-cis-9,12-octadecadienoic acid (linoleic acid); all-cis-6,9,12-octadecatrienoic acid (gamma-linolenic acid, GLA); all-cis-11,14-eicosadienoic acid (eicosadienoic acid); all-cis-8,11,14-eicosatrienoic acid (dihomo-gamma-linolenic acid, DGLA); all-cis-5,8,11,14-eicosatetraenoic acid (arachidonic acid, AA); all-cis-13,16-docosadienoic acid (docosadienoic acid); all-cis-7,10,13,16-docosatetraenoic acid (adrenic acid); and all-cis-4,7,10,13,16-docosapentaenoic acid (docosapentaenoic acid). In yet another alternative embodiment, the PUFA may be an omega-9 fatty acid in which the first double bond occurs in the ninth carbon-carbon bond from the methyl end of the carbon chain, or a conjugated fatty acid, in which at least one pair of double bonds are separated by only one single bond. Suitable examples of omega-9 fatty acids include cis-9-octadecenoic acid (oleic acid); cis-11-eicosenoic acid (eicosenoic acid); all-cis-5,8,11-eicosatrienoic acid (mead acid); cis-13-docosenoic acid (erucic acid), and cis-15-tetracosenoic acid (nervonic acid). Examples of conjugated fatty acids include 9Z,11 E-octadeca-9,11-dienoic acid (rumenic acid); 10E,12Z-octadeca-9,11-dienoic acid; 8E,10E,12Z-octadecatrienoic acid (α-calendic acid); 8E,10E,12E-octadecatrienoic acid (β-Calendic acid); 8E,10Z,12E-octadecatrienoic acid (jacaric acid); 9E,11E,13Z-octadeca-9,11,13-trienoic acid (α-eleostearic acid); 9E,11E,13E-octadeca-9,11,13-trienoic acid (β-eleostearic acid); 9Z,11Z,13E-octadeca-9,11,13-trienoic acid (catalpic acid), and 9E,11Z,13E-octadeca-9,11,13-trienoic acid (punicic acid).

In additional embodiments, the bioactive agent may be one or more essential oils. Suitable essential oils include, but are not limited to, peppermint oil, cinnamon leaf oil, lemongrass oil, clove oil, castor oil, wintergreen oil, sweet orange, spearmint oil, cederwood oil, aldehyde C₁₆, α terpineol, amyl cinnamic aldehyde, amyl salicylate, anisic aldehyde, benzyl alcohol, benzyl acetate, camphor, capsaicin, cinnamaldehyde, cinnamic alcohol, carvacrol, carveol, citral, citronellal, citronellol, p cymene, diethyl phthalate, dimethyl salicylate, dipropylene glycol, eucalyptol (cineole), eugenol, iso-eugenol, galaxolide, geraniol, guaiacol, ionone, listea cubea, menthol, menthyl salicylate, methyl anthranilate, methyl ionone, methyl salicylate, a phellandrene, pennyroyal oil, perillaldehyde, 1 or 2 phenyl ethyl alcohol, 1 or 2 phenyl ethyl propionate, piperonal, piperonyl acetate, piperonyl alcohol, D pulegone, terpinen 4 ol, terpinyl acetate, 4 tert butylcyclohexyl acetate, thyme oil, thymol, metabolites of trans-anethole, vanillin, ethyl vanillin, derivatives thereof, or combinations thereof.

In still other embodiments, the bioactive agents may be one or more probiotics or prebiotics. Probiotics and prebiotics include agents derived from yeast or bacteria that promote good digestive health. By way of non-limiting example, yeast-derived probiotics and prebiotics include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. In an exemplary embodiment, the yeast-derived agent may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae. Probiotics and prebiotics may also include bacteria cell wall derived agents such as peptidoglycan and other components derived from gram-positive bacteria with a high content of peptidoglycan. Exemplary gram-positive bacteria include Lactobacillus acidophilus, Bifedobact thermophilum, Bifedobat longhum, Streptococcus faecium, Bacillus pumilus, Bacillus subtilis, Bacillus licheniformis, Lactobacillus acidophilus, Lactobacillus casei, Enterococcus faecium, Bifidobacterium bifidium, Propionibacterium acidipropionici, Propionibacteriium freudenreichii, and Bifidobacterium pseudolongum.

In alternate embodiments, the bioactive agent may be one or more enzymes or enzyme variants. Suitable non-limiting examples of enzymes include amylases, carbohydrases, cellulases, esterases, galactonases, galactosidases, glucanases, hemicellulases, hydrolases, lipases, oxidoreductases, pectinases, peptidases, phosphatases, phospholipases, phytases, proteases, transferases, xylanases, or combinations thereof.

In further embodiments, the bioactive agent may be one or more herbals. Suitable herbals and herbal derivatives, as used herein, refer to herbal extracts, and substances derived from plants and plant parts, such as leaves, flowers, and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, scullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca, or combinations thereof.

In still other embodiments, the bioactive agent may be one or more natural pigments. Suitable pigments include, without limit, actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, γ-carotine, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene, or combinations thereof.

In yet other embodiments, the bioactive agent may be one or more antibiotics approved for use in livestock and poultry (i.e., antibiotics not considered critical or important for human health). Non-limiting examples of approved antibiotics include bacitracin, carbadox, ceftiofur, enrofloxacin, florfenicol, laidlomycin, linomycin, oxytetracycline, roxarsone, tilmicosin, tylosin, and virginiamycin.

(b) Feed

A further aspect of the present disclosure encompasses an animal feed ration comprising the compositions described in Section (I) or a premix or supplement as described in Section (II)(a).

Feed ingredients that may be utilized in the present disclosure to satisfy an animal's maintenance energy requirements may include feed ingredients that are commonly provided to animals for consumption. Examples of such feed ingredients include grains, forage products, feed meals, feed concentrates, and the like.

Suitable grains include corn, corn gluten meal, soybeans, soybean meal, wheat, barley, oats, sorghum, rye, rice, and other grains, and grain meals.

Forage products are feed ingredients such as vegetative plants in either a fresh (pasture grass or vegetation), dried, or ensiled state and may incidentally include minor proportions of grain (e.g., kernels of corn that remain in harvested corn plant material after harvest). Forage includes plants that have been harvested and optionally fermented prior to being provided to ruminants as a part of their diet. Thus, forage includes hay, haylage, and silage. Examples of hay include harvested grass, either indigenous to the location of the ruminants being fed or shipped to the feeding location from a remote location. Non-limiting examples of hay include alfalfa, Bermuda grass, bahia grass, limpo grass, rye grass, wheat grass, fescue, clover, and the like as well as other grass varieties that may be native to the location of the ruminants being provided the ruminant feed ration.

It is beneficial if the forage is relatively high quality (i.e., contains relatively levels of metabolizable nutrients which permit the animal to satisfy its nutrient and maintenance energy requirements before reaching its consumption capacity). If the forage is of low quality, the animal may not metabolize it adequately to achieve desired performance effects (e.g., satisfy its nutrient and/or maintenance energy requirements), not only compromising the nutritional benefit from the forage per se, but also causing the animal to feel full or bloated, and possibly deterring it from consuming sufficient nutrients.

Haylage is a forage product that has been naturally fermented by harvesting a hay crop while the sap is still in the plant. The harvested hay or hay bales are then stored in an air-tight manner in which fermentation can occur. The fermentation process converts the sugars in the plants into acids which lower the pH of the harvested hay and preserves the forage.

Silage, similar to haylage, is a forage product that is produced from the harvest, storage and fermentation of green forage crops such as corn and grain sorghum plants. These crops are chopped, stems and all, before the grain is ready for harvest. The plant material is stored in silos, storage bags, bunkers, or covered piles causing the material to ferment, thereby lowering the pH and preserving the plant material until it can be fed.

Forage products also include high fiber sources and scrap vegetation products such as green chop, corncobs, plant stalks, and the like.

Feed concentrates are feedstuffs that are high in energy and low in crude fiber. Concentrates also include a source of one or more ingredients that are used to enhance the nutritional adequacy of a feed supplement mix, such as vitamins and minerals.

The feed may be supplemented with a fat source. Non-limiting fats include plant oils, fish oils, animal fats, yellow grease, fish meal, oilseeds, distillers' grains, or combinations thereof. The fat source will generally comprise from about 1% to about 10% of the dry mass of the total feed ration, more preferably from about 2% to about 6%, and most preferably from about 3% to about 4%.

As used herein, “piglet feed rations” generally refer to feed rations provided to piglets from the time of weaning to about the grower/finisher stage. In this context, the term generally refers to the feed ration provided to pigs that are from about three weeks of age to about seven weeks of age. Generally speaking, piglet feed rations comprise two distinct phases: Phase I includes feed rations fed to piglets from about one day to about ten days post weaning, and Phase II includes feed rations fed to piglets from about ten day to about twenty-one days post weaning.

Common ingredients in piglet feed rations typically comprise grains (e.g., corn, barley, grain sorghum, oats, soybeans, wheat, etc.), crude proteins (e.g., fish meal, gluten meal, meat meal, soybean meal, tankage, which is the residue that remains after rendering fat in a slaughterhouse, etc.), crude fat (e.g., fish oils, vegetable oils, animal fats, yellow grease, etc.), supplemental amino acids (e.g., lysine, methionine or methionine analogs, etc.), vitamins, minerals, mycotoxin inhibitors, antifungal agents, and the like. Phase I formulations generally comprise from about 15% to about 30% by weight lactose. Phase II formulations generally comprise from about 4% to about 12% by weight lactose.

Other ingredients may be optionally included in the animal feed to provide additional nutrients to the animals. Examples of optional ingredients include vitamins, minerals, and the like (see Section (I)(a)). These ingredients may also be excluded as necessary to provide a feed ration to animals that can be tailored to meet their nutritional needs.

(c) Feed Rations

Feed rations of the present disclosure typically are formulated to meet the nutrient and energy demands of a particular animal. The nutrient and energy content of many common animal feed ingredients have been measured and are available to the public. The National Research Council has published books that contain tables of common ruminant feed ingredients and their respective measured nutrient and energy content. Additionally, estimates of nutrient and maintenance energy requirements are provided for growing and finishing cattle according to the weight of the cattle. National Academy of Sciences, Nutrient Requirements of Beef Cattle, Appendix Tables 1-19, 192-214, (National Academy Press, 2000); Nutrient Requirements of Dairy Cattle (2001), which are each incorporated herein by their entirety. This information can be utilized by one skilled in the art to estimate the nutritional and maintenance energy requirements of animal and determine the nutrient and energy content of animal feed ingredients.
(III) Methods of Improving Dietary Phosphorus and Calcium Utilization in Animals

Yet another aspect of the present disclosure encompasses methods of using the compositions of the disclosure for improving dietary utilization of phosphorous and calcium in an animal. In particular, the methods comprise administering to the animal a composition comprising a metal chelate of Formula (I): LₓM^{y} and a phytase, wherein L is ligand, M is a metal ion, and x and y are integers from 1 to 10. The animal administered the metal chelate and phytase has increased utilization of dietary phosphorous and calcium relative to an animal administered an inorganic salt of the metal and the phytase (see Example 1).

In addition, another aspect of the present disclosure encompasses methods for improving dietary phosphorus and calcium utilization in an animal fed a diet comprising a phytase, wherein the methods comprise administering to the animal a metal chelate metal rather than an inorganic salt of the metal.

Providing the composition as described in Section (I) to an animal improves dietary phosphorus and calcium utilization. Increased phosphorus and calcium utilization may lead to increase growth and performance of an animal. In this context, the composition as described in Section (I), when fed in a feed ration to an animal, may increase digestibility of feed comprising phosphorus and calcium.

In an embodiment, the methods provided herein may increase phosphorus utilization in an animal about 10, about 15, about 20, about 30, about 40, about 45, about 50, about 55, or about 60% as compared to feeding an animal a ration without the compositions as described in Section (I).

In an embodiment, the methods provided herein may increase bone phosphorus levels in an animal about 1, about 2, about 3, about 4, about 5, about 6, about 7, about 8, about 9, about 10, about 15, or about 15% (DM basis) as compared to feeding an animal a ration without the compositions as described in Section (I).

In an embodiment, the methods provided herein may increase calcium utilization in an animal about 10, about 15, about 20, about 30, about 40, about 45, about 50, about 55, about 60, about 65, about 70, or about 75% as compared to feeding an animal a ration without the compositions as described in Section (I).

In an embodiment, the methods provided herein may increase bone calcium levels in an animal about 1, about 2, about 3, about 4, about 5, about 6, about 7, about 8, about 9, about 10, about 15, about 20, or about 25% (DM basis) as compared to feeding an animal a ration without the compositions as described in Section (I).

In an embodiment, the methods provided herein may increase weight gain in an animal of about 5, about 10, about 15, about 20, about 30, about 40, about 45, or about 50% as compared to feeding an animal a ration without the compositions as described in Section (I).

The compositions as described in Section (I) may reduce the antagonism of phytases by metal salts in the feed.

The amount of the compositions described in Section (I) can and will vary depending upon the type of animal and age of the animal. In general, the amount of metal administered to the animal as the metal chelate will range from about 10 to about 200 ppm as calculated based on the weight of the diet. In an embodiment, the amount of metal administered as chelate may be about 10, about 20, about 30, about 40, about 50, about 60, about 70, about 80, about 90, about 100, about 110, about 120, about 130, about 140, about 150, about 160, about 170, about 180, about 190, or about 200 ppm. In an exemplary embodiment, the amount of metal administered as the metal chelate may be about 80 ppm. In another exemplary embodiment, the amount of metal administered as the chelate may be about 100 ppm. In still exemplary embodiment, the amount of metal administered as the chelate may be about 150 ppm.

The amount of phytase administered in the composition can and will vary depending upon the type of animal and age of the animal. In an embodiment, the amount of phytase administered the animal may be from about 100 to about 1000 FTU per kg of animal feed. In a further embodiment, the amount of phytase administered to the animal may be from about 200 to about 900, about 300 to about 800, about 400 to about 700 FTU per kg. In other embodiments, the amount of phytase administered to the animal may be about 100, about 150, about 200, about 250, about 300, about 350, about 400, about 450, about 500, about 550, about 600, about 650, about 700, about 750, about 800, about 850, about 900, about 950, or about 1000 FTU per kg of animal feed. In an exemplary embodiment, the amount of phytase administered to the animal may be about 500 FTU per kg of animal feed.

(a) Animal

Suitable animals may include, but are not limited to, livestock animals, companion animals, lab animals, and zoological animals. In one embodiment, the animal may be a rodent, e.g. a mouse, a rat, a guinea pig, hamster, etc. In another embodiment, the animal may be a livestock animal. Non-limiting examples of suitable livestock animals may include pigs, cows, goats, sheep, llamas, alpacas, etc. In yet another embodiment, the subject may be a companion animal. Non-limiting examples of companion animals may include pets such as dogs, cats, horses, rabbits, and birds. In yet another embodiment, the subject may be a zoological animal. As used herein, a “zoological animal” refers to an animal that may be found in a zoo. Such animals may include non-human primates, large cats, wolves, bears, hippos, kangaroos, etc. In still another embodiment, the animal may be a laboratory animal. Non-limiting examples of a laboratory animal may include rodents, canines, felines, and non-human primates. In an embodiment, the animal may be a ruminant. In a preferred embodiment, the animal may be a non-ruminant. In another preferred embodiment, the animal may be a monogastric animal. In a further preferred embodiment, the animal may be a livestock animal. In an exemplary embodiment, the animal may be a pig, or poultry such as chicken, turkey, or duck.

### Enumerated Embodiments

The following enumerated embodiments are presented to illustrate certain aspects of the present invention, and are not intended to limit its scope.

1. A composition comprising a metal chelate and a phytase, wherein the composition comprises about 50 ppm to about 1800 ppm of the metal chelate and about 6 ppm to about 1000 ppm of the phytase, and the metal chelate comprises at least one metal ion and at least one ligand of Formula (Ill):

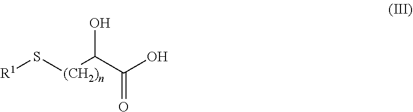

wherein, n is an integer from 1 to 5; and R¹ is C₁ to C₆ alkyl or C₁ to C₆ substituted alkyl.

2. The composition of embodiment 1, wherein R¹ is methyl or ethyl, and n is 1 or 2.

3. The composition of embodiments 1 or 2, wherein R¹ is methyl and n is 2.

4. The composition of any one of embodiments 1 to 3, wherein the metal ion is selected from the group consisting of calcium, chromium, cobalt, copper, iron, magnesium, manganese, nickel, potassium, sodium, and zinc.

5. The composition of embodiment 4, wherein the metal ion is selected from the group consisting of zinc, iron, copper, and manganese.

6. The composition of any one of embodiments 1 to 5, wherein the ratio of the ligand to the metal ion is from about 1:1 to about 3:1.

7. The composition of embodiment 6, wherein the ratio of the ligand to the metal ion is about 2:1.

8. The composition of any one of embodiments 1 to 7, wherein the ligand is 2-hydroxy-4-(methylthio)butanoic acid (HMTBA) and the metal ion is copper.

9. The composition of any one of embodiments 1 to 7, wherein the ligand is HMTBA and the metal ion is zinc.

10. The composition of any one of embodiments 1 to 7, wherein the ligand is HMTBA and the metal ion is manganese.

11. The composition of any one of embodiments 1 to 10, wherein the phytase is of fungal, yeast, or bacterial origin.

12. The composition of any one of embodiments 1 to 11, wherein the phytase is recombinantly produced.

13. The composition of any one of embodiments 1 to 12, wherein the composition comprises about 60 ppm to 1600 ppm of the metal chelate.

14. The composition of any one of embodiments 1 to 13, wherein the composition comprises about 8 ppm to about 800 ppm of the phytase.

15. The composition of any one of embodiments 1 to 14, the composition comprises about 5 to about 300 ppm of the metal ion.

16. The composition of embodiment 15, wherein the composition comprises about 10 ppm to about 200 ppm of the metal ion.

17. A method for improving dietary phosphorus and calcium utilization in an animal, the method comprising administering to the animal a metal chelate and a phytase, wherein dietary phosphorus and calcium utilization is increased relative to administration of an inorganic salt of the metal and the phytase.

18. The method of embodiment 17, wherein the metal chelate has Formula (I):

LₓM^{y} (I)

wherein, L is a ligand; M is a metal ion; and x and y are integers from 1 to 10.

19. The method of embodiment 18, wherein L is an organic acid moiety, an amino acid moiety, or a derivative thereof.

20. The method of embodiment 19, wherein the organic acid moiety is selected from the group consisting of adipate, ascorbate, caprylate, citrate, furmarate, glucoheptonate, gluconate, glutarate, glycerophosphate, lactate, ketoglutarate, malate, malonate, orotate, oxlate, pantothenate, picolinate, pidolate, sebacate, succinate, and tartrate; and the amino acid moiety is selected from the group consisting of alanate, arginate, asparaginate, aspartate, cysteinate, glutaminate, glutamate, histidinate, homocysteinate, isoleucinate, lysinate, methionate, phenylalinate, prolinate, serinate, threonate, typtophanate, tyrosinate, and valinate.

21. The method of embodiment 18, wherein L is a compound of Formula (II):

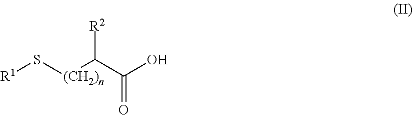

wherein, n is an integer from 1 to 5; R¹ is C₁ to C₆ alkyl or C₁ to C₆ substituted alkyl; and R² is hydroxyl or amino.

22. The method of embodiment 21, wherein R¹ is methyl or ethyl, and n is 1 or 2.

23. The method of embodiments 21 or 22, wherein n is 2, R¹ is methyl, and R² is hydroxyl.

24. The method of any one of embodiments 17 to 23, wherein the metal ion is selected from the group consisting of calcium, chromium, cobalt, copper, iron, magnesium, manganese, nickel, potassium, sodium, and zinc.

25. The method of embodiment 24, wherein the metal ion is selected from the group consisting of zinc, iron, copper, and manganese.

26. The method of any one of embodiments 17 to 25, wherein x is from 1 to 5.

27. The method of any one of embodiments 18 to 26, wherein y is from 1 to 5.

28. The method of embodiments 26 or 27, wherein each of x and y is from 1 to 3.

29. The method of embodiment 28, wherein each of x and y is 2.

30. The method of any one of embodiments 18 to 29, wherein the ligand is 2-hydroxy-4-(methylthio)butanoic acid (HMTBA) and the metal ion is copper.

31. The method of any one of embodiments 18 to 29, wherein the ligand is HMTBA and the metal ion is zinc.

32. The method of any one of embodiments 18 to 29, wherein the ligand is HMTBA and the metal ion is manganese.

33. The method of any one of embodiments 17 to 32, wherein the phytase is of fungal, yeast, or bacterial origin.

34. The method of any one of embodiments 17 to 33, wherein the amount of metal administered to the animal as the metal chelate is from about 10 to about 200 ppm.

35. The method of embodiments 34, wherein the amount of the metal administered to the animal is from about 50 to about 150 ppm.

36. The method of embodiments 35, wherein the amount of the metal administered to the animal is from about 80 to about 100 ppm.

37. The method of any one of embodiments 17 to 36, wherein the amount of the phytase administered to the animal is from about 100 to about 1000 phytase units (FTU) per kg.

38. The method of embodiment 37, wherein the amount of the phytase is from about 400 to about 600 FTU per kg.

39. The method of any one of embodiments 17 to 38, wherein the animal is a ruminant animal or a non-ruminant animal.

40. The method of any one of embodiments 17 to 39, wherein the animal is a monogastric animal.

41. The method of embodiment 40, wherein the monogastric animal is a pig, poultry, or horse.

42. A method for improving dietary phosphorus and calcium utilization in an animal fed a diet comprising a phytase, the method comprising administering to the animal a metal chelate rather than an inorganic salt of the metal.

43. The method of embodiment 42, wherein the metal chelate has Formula (I):

LₓM^{y} (I)

wherein, L is a ligand; M is a metal ion; and X and y are integers from 1 to 10.

44. The method of embodiment 43, wherein L is an organic acid moiety, an amino acid moiety, or a derivative thereof.

45. The method of embodiment 44, wherein the organic acid moiety is selected from the group consisting of adipate, ascorbate, caprylate, citrate, furmarate, glucoheptonate, gluconate, glutarate, glycerophosphate, lactate, ketoglutarate, malate, malonate, orotate, oxlate, pantothenate, picolinate, pidolate, sebacate, succinate, and tartrate; and the amino acid moiety is selected from the group consisting of alanate, arginate, asparaginate, aspartate, cysteinate, glutaminate, glutamate, histidinate, homocysteinate, isoleucinate, lysinate, methionate, phenylalinate, prolinate, serinate, threonate, typtophanate, tyrosinate, and valinate.

46. The method of embodiment 43, wherein L is a compound of Formula (II):

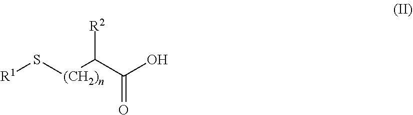

wherein, n is an integer from 1 to 5; R¹ is C₁ to C₆ alkyl or C₁ to C₆ substituted alkyl; and R² is hydroxyl or amino.

47. The method of embodiment 46, wherein R¹ is methyl or ethyl, and n is 1 or 2.

48. The method of embodiments 46 or 47, wherein n is 2, R¹ is methyl, and R² is hydroxyl.

49. The method of any one of embodiments 42 to 48, wherein the metal ion is selected from the group consisting of calcium, chromium, cobalt, copper, iron, magnesium, manganese, nickel, potassium, sodium, and zinc.

50. The method of embodiment 49, wherein the metal ion is selected from the group consisting of zinc, iron, copper, and manganese.

51. The method of any one of embodiments 42 to 50, wherein x is from 1 to 5.

52. The method of any one of embodiments 43 to 51, wherein y is from 1 to 5.

53. The method of embodiments 51 or 52, wherein each of x and y is from 1 to 3.

54. The method of embodiment 53, wherein each of x and y is 2.

55. The method of any one of embodiments 43 to 54, wherein the ligand is 2-hydroxy-4-(methylthio)butanoic acid (HMTBA) and the metal ion is copper.

56. The method of any one of embodiments 43 to 54, wherein the ligand is HMTBA and the metal ion is zinc.

57. The method of any one of embodiments 43 to 54, wherein the ligand is HMTBA and the metal ion is manganese.

58. The method of any one of embodiments 42 to 57, wherein the phytase is of fungal, yeast, or bacterial origin.

59. The method of any one of embodiments 42 to 58, wherein the amount of the metal administered to the animal as the metal chelate is from about 10 to about 200 ppm.

60. The method of embodiments 59, wherein the amount of the metal administered to the animal is from about 50 to about 150 ppm.

61. The method of embodiment 60, wherein the amount of the metal administered to the animal is from about 80 to about 100 ppm.

62. The method of any one of embodiments 42 to 61, wherein the animal is a ruminant animal or a non-ruminant animal.

63. The method of any one of embodiments 42 to 61, wherein the animal is a monogastric animal.

64. The method of embodiment 63, wherein the monogastric animal is a pig, poultry, or horse.

### Definitions

When introducing elements of the embodiments described herein, the articles “a,” “an,” “the,” and “said” are intended to mean that there are one or more of the elements. The terms “comprising,” “including,” and “having” are intended to be inclusive and mean that there may be additional elements other than the listed elements.

The term “lipids,” as used herein, refers to a substance that is water insoluble, but soluble in organic solvents (e.g., ether, chloroform, hexane, etc.). One example of a simple lipid is triglycerides. Triglycerides are found primarily in cereal grains, oilseeds, and animal fats. The basic structure of triglycerides consists of one unit of glycerol and three units of fatty acids.

The term “nutrient,” as used herein, refers to chemical substances that are generally necessary for one or more of the maintenance, growth, production, reproduction and/or health of the ruminant. By way of non-limiting example, nutrients include water, energy (e.g., carbohydrates, proteins, and lipids), proteins (e.g., nitrogenous compounds), minerals, and vitamins.

The term “ruminant,” as used herein is meant to encompass mature and immature animals with multi-compartment stomachs, including but not limited to, cattle, sheep, deer, goats, musk, ox, buffalo, giraffe, and camels. For example, cattle and sheep have a stomach with four compartments comprising the rumen, reticulum, omasum and abomasum.

The term “non-ruminant,” as used herein is meant to encompass mature and immature animals with a single stomach compartment, including but not limited to, rats, dogs, cats, rabbits, pigs, and horses.

The abbreviation “ppm,” as used herein, stands for parts per million.

The abbreviation “DM,” as used herein, stands for dry matter.

The abbreviation “ATTD,” as used herein, stands for apparent total tract digestibility.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Chelated Zn and Cu Improved Phosphorus Utilization in the Presence of phytase Supplementation Compared with Inorganic Zn and Cu

### Introduction

This study aimed to investigate the effect of Zn sources (Zn(HMTBA)₂ vs. ZnO) or Cu sources (Cu(HMTBA)₂ vs. CuSO₄) and phytase on the calcium and phosphorus digestibility in nursery pigs.

### Methods

A total of 288 weaning barrows [TR-4×PIC C-22, initial body weight (BW)=5.71±0.71 kg] were allotted to 1 of 8 dietary treatments according to randomized complete block design. Pigs were blocked by initial body weight. The study started from weaning (day 0) to day 42 post-weaning. Three-phase feeding program was used in this study, in which day 0 to 14, day 15 to 28, and day 29 to 42 were considered as nursing phase 1, 2, and 3, respectively. A basal diet for each phase was formulated to meet the energy and nutrient requirement for different stages of pigs according to previously published recommendation,¹ with the exception of P [0.15% reduction of standardized total tract digestible (STTD) P] and Ca (adjusted according to the fixed ratio of Ca/STTD P=2.15). Eight experimental diets were formulated based on basal diet supplemented with phytase (0 or 500 FTU/kg), Zn sources (2000 ppm Zn from ZnO in phase 1 and 2, 100 ppm Zn from ZnO in phase 3; 100 ppm Zn from Zn(HMTBA)₂ from phase 1 to 3) and Cu sources (150, 80, 80 ppm Cu from CuSO₄ or Cu(HMTBA)₂ in phase 1, 2, and 3, respectively) in a 2×2×2 factorial treatment design. Titanium dioxide at 0.4% was included in phase 2 diets to measure Ca and P digestibility. The dietary treatments are displayed in Table 1, Table 2, and Table 3.

TABLE 1
General characteristics of dietary treatments during nursing phase 1 (d 0-14)

| | | | Zn level | | Cu level | Phytase |
|---|---|---|---|---|---|---|
| | Treatment description | Zn source | (ppm) | Cu Source | (ppm) | (FTU/kg) |
| A | ZnO + CuSO₄ | ZnO | 2000 | CuSO₄ | 150 | — |
| B | ZnO + Cu(HMTBA)₂ | ZnO | 2000 | Cu(HMTBA)₂ | 150 | — |
| C | Zn(HMTBA)₂ + CuSO₄ | Zn(HMTBA)₂ | 100 | CuSO₄ | 150 | — |
| D | Zn(HMTBA)₂ + | Zn(HMTBA)₂ | 100 | Cu(HMTBA)₂ | 150 | — |
| | Cu(HMTBA)₂ | | | | | |
| E | ZnO + CuSO₄ + Phytase | ZnO | 2000 | CuSO₄ | 150 | 500 |
| F | ZnO + Cu(HMTBA)₂ + | ZnO | 2000 | Cu(HMTBA)₂ | 150 | 500 |
| | Phytase | | | | | |
| G | Zn(HMTBA)₂ + CuSO₄ + | Zn(HMTBA)₂ | 100 | CuSO₄ | 150 | 500 |
| | Phytase | | | | | |
| H | Zn(HMTBA)₂ + | Zn(HMTBA)₂ | 100 | Cu(HMTBA)₂ | 150 | 500 |
| | Cu(HMTBA)₂ + Phytase | | | | | |

TABLE 2
Genderal characteristics of dietary treatments during nursery phase 2 (d 15-28)

| | | | Zn level | | Cu level | Phytase |
|---|---|---|---|---|---|---|
| | Treatment description | Zn source | (ppm) | Cu source | (ppm) | (FTU/kg) |
| A | ZnO + CuSO4 | ZnO | 2000 | CuSO₄ | 80 | — |
| B | ZnO + Cu(HMTBA)₂ | ZnO | 2000 | Cu(HMTBA)₂ | 80 | — |
| C | Zn(HMTBA)₂ + CuSO₄ | Zn(HMTBA)₂ | 100 | CuSO₄ | 80 | — |
| D | Zn(HMTBA)₂ + | Zn(HMTBA)₂ | 100 | Cu(HMTBA)₂ | 80 | — |
| | Cu(HMTBA)₂ | | | | | |
| E | ZnO + CuSO₄ + Phytase | ZnO | 2000 | CuSO₄ | 80 | 500 |
| F | ZnO + Cu(HMTBA)₂ + | ZnO | 2000 | Cu(HMTBA)₂ | 80 | 500 |
| | Phytase | | | | | |
| G | Zn(HMTBA)₂ + CuSO₄ + | Zn(HMTBA)₂ | 100 | CuSO₄ | 80 | 500 |
| | Phytase | | | | | |
| H | Zn(HMTBA)₂ + | Zn(HMTBA)₂ | 100 | Cu(HMTBA)₂ | 80 | 500 |
| | Cu(HMTBA)₂ + Phytase | | | | | |

TABLE 3
General characteristics of dietary treatments during nursery phase 3 (d 29-42)

| | | | Zn level | | Cu level | Phytase |
|---|---|---|---|---|---|---|
| | Treatment description | Zn source | (ppm) | Cu source | (ppm) | (FTU/kg) |
| A | ZnO + CuSO₄ | ZnO | 100 | CuSO₄ | 80 | — |
| B | ZnO + Cu(HMTBA)₂ | ZnO | 100 | Cu(HMTBA)₂ | 80 | — |
| C | Zn(HMTBA)₂ + CuSO₄ | Zn(HMTBA)₂ | 100 | CuSO₄ | 80 | — |
| D | Zn(HMTBA)₂ + | Zn(HMTBA)₂ | 100 | Cu(HMTBA)₂ | 80 | — |
| | Cu(HMTBA)₂ | | | | | |
| E | ZnO + CuSO₄ + Phytase | ZnO | 100 | CuSO₄ | 80 | 500 |
| F | ZnO + Cu(HMTBA)₂ + | ZnO | 100 | Cu(HMTBA)₂ | 80 | 500 |
| | Phytase | | | | | |
| G | Zn(HMTBA)₂ + CuSO₄ + | Zn(HMTBA)₂ | 100 | CuSO₄ | 80 | 500 |
| | Phytase | | | | | |
| H | Zn(HMTBA)₂ + | Zn(HMTBA)₂ | 100 | Cu(HMTBA)₂ | 80 | 500 |
| | Cu(HMTBA)₂ + Phytase | | | | | |

### Sample Collection

Fresh fecal samples were collected via grab sampling from each pig per pen at least twice per day from day 24 to 26. Fecal samples collected from the same pen during the 3 days were pooled together, and a subsample was placed inside a large oven set at 65° C. Oven dried fecal samples were stored at room temperature until analysis for dry matter (DM), Ca, P, and titanium (Ti).

On day 42, 2 pigs from each pen were euthanized to collect the right front legs. The third metacarpal bone from each right front leg was used to determine DM, Ca, P, and ash concentrations.

### Calculation

The apparent total tract digestibility (ATTD) coefficient for Ca and P in each treatment was calculated using the following formula:

ATTD of Ca, %=[1−(Ca_{feces}/Ca_{diet})×(M_{diet}/M_{feces})]×100 (Equation 1)

ATTD of P, %=[1−(P_{feces}/P_{diet})×M_{diet}/M_{feces})]×100 (Equation 2)

where Ca_{feces} and Ca_{diet} represented Ca concentrations (g/kg) in feces and diet DM, respectively; P_{feces} and P_{diet} represented P concentrations (g/kg) in feces and diet DM; M_{diet} and M_{digesta} represent the marker concentrations (g/kg) in diet and feces DM, respectively.

The standard total tract digestibility (STTD) of Ca was calculated according to the following equation:

STTD of Ca, %=[ATTD of Ca+(basal ECaL_{end}/Ca_{diet})×100] (Equation 3)

where Ca_{diet} represented Ca concentrations (g/kg) in diet DM, respectively; basal ECaL_{end} represented basal endogenous loss of Ca which was approximately 330 mg/kg DMI.

The standard total tract digestibility (STTD) of P was calculated according to the following equation:

STTD of P, %=[ATTD of P+(basal EPL_{end}/P_{diet})×100] (Equation 4)

where P_{diet} represented P concentrations (g/kg) in diet DM, respectively; basal EPL_{end} represented basal endogenous loss of P which was approximately 190 mg/kg DMI.

### Statistical Analysis

SAS 9.4 (SAS Inst. Inc., Gary, N.C.) was used for all data analysis. Pen served as the experimental unit. The LSMEANS statement was be used to calculate the least square means. Tukey-Kramer adjustment was used for multiple comparisons of the least square means. Pooled SEM will be calculated for each measurement. A probability of P 0.05 will be considered as significant and 0.05<P≤0.10 will be declared as a trend. Mineral sources (Zn and Cu), phytase and their interactions were considered as the main effect, while block was considered as the random effect.

TABLE 4
Effect of Zn sources (Zn(HMTBA)₂ vs. ZnO) and phytase
on standardized total tract digestibility of Ca and P

| | | | | | | P values | | |
|---|---|---|---|---|---|---|---|---|
| Items | No phytase | | With phytase¹ | | | | | Zn source × |
| STTD², % | ZnO | Zn(HMTBA)₂ | ZnO | Zn(HMTBA)₂ | SEM | Zn source | Phytase | phytase |
| Ca | 48.49 | 53.30 | 59.18 | 65.63 | 2.69 | <0.01 | <0.01 | 0.56 |
| P | 2.67 | 32.65 | 33.68 | 46.36 | 2.63 | <0.01 | <0.01 | <0.01 |

¹Phytase inclusion level is 500 FTU/kg.
²STTD represents standardized total tract digestibility.

TABLE 5
Effect of Cu sources (Cu(HMTBA)₂ vs. CuSO₄) and phytase on gain to feed ratio
during d 15 to 28, bone Ca, P and ash concentrations in nursery pigs

| | | | | | | P values | | |
|---|---|---|---|---|---|---|---|---|
| | No phytase | | With phytase¹ | | | Cu | | Cu source × |
| Items | CuSO₄ | Cu(HMTBA)₂ | CuSO | Cu(HMTBA)₂ | SEM | source | Phytase | phytase |
| G:F² (d 15 to 28), g/g | 0.76 | 0.75 | 0.80 | 0.84 | 0.02 | 0.29 | <0.01 | 0.09 |
| Bone Ca (DM basis), % | 13.04 | 12.55 | 14.14 | 14.89 | 0.18 | 0.62 | <0.01 | 0.02 |
| Bone P (DM basis), % | 6.65 | 6.44 | 7.38 | 7.81 | 0.10 | 0.41 | <0.01 | 0.03 |
| Bone ash (DM basis), % | 36.69 | 35.95 | 40.41 | 41.97 | 0.49 | 0.58 | <0.01 | 0.10 |

¹Phytase inclusion level is 500 FTU/kg.
²G:F represents gain to feed ratio.

Results

Zn at 100 ppm significantly (P<0.01, Table 4) increased STTD of Ca without phytase supplementation (53.30% vs. 48.48%) or with phytase supplementation (65.63% vs. 59.18%), compared with ZnO at 2000 ppm. Phytase supplementation also significantly (P<0.01) increased STTD of Ca. However, there was no interaction between Zn sources and phytase in terms of STTD of Ca. The absolute increase of STTD of Ca for Zn(HMTBA)₂ compared with ZnO was 4.81% and 6.45% for without phytase and with phytase supplementation conditions, respectively.

Zn(HMTBA)₂ at 100 ppm significantly (P<0.01) increased STTD of P without phytase supplementation (2.67% vs. 32.65%) or with phytase supplementation (33.68% vs. 46.36%), compared with ZnO at 2000 ppm. Phytase supplementation also significantly (P<0.01) increased STTD of P. Additionally, there was a significant (P<0.01) interaction between Zn sources and phytase in terms of STTD of P. The absolute increase of STTD of P for Zn(HMTBA)₂ compared with ZnO was 29.98% without phytase and 12.68% with phytase supplementation conditions, respectively, indicating that Zn(HMTBA)₂ could increase phytase efficacy even though the magnitude of improvement of P digestibility by Zn(HMTBA)₂ was reduced as compared with that of ZnO.

There were no significant differences between Cu(HMTBA)₂ and CuSO4 when both were supplemented at 80 ppm in terms of gain to feed ratio (G:F) during day 15 to 28 (P=0.29, Table 5), bone Ca (P=0.62), P (P=0.41) and ash (P=0.58). However, phytase supplementation significantly (P<0.01) improved gain to feed ratio, bone Ca, P, and ash, compared with no phytase supplementation group. Additionally, the improvement in G:F during d 15 to 28 (P =0.09) and bone ash (P=0.10) for Cu(HMTBA)₂ compared with CuSO₄ only showed tendencies in the presence of phytase. Furthermore, the improvement in bone Ca (P=0.02) and P (P=0.03) concentrations for Cu(HMTBA)₂ compared with CuSO₄ only showed significant difference in the presence of phytase. These results indicated that Cu(HMTBA)₂ can improve phytase efficacy to increase Ca and P utilization in pigs, compared with CuSO₄.

All publications, patents, patent applications and other references cited in this application are herein incorporated by reference in their entirety as if each individual publication, patent, patent application or other reference were specifically and individually indicated to be incorporated by reference.

What is claimed is:

1. A composition comprising a metal chelate and a phytase, wherein the composition comprises about 50 ppm to about 1800 ppm of the metal chelate and about 6 ppm to about 1000 ppm of the phytase, and the metal chelate comprises at least one metal ion and at least one ligand of Formula (Ill): 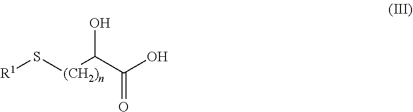
wherein:
n is an integer from 1 to 5; and
R¹ is C₁ to C₆ alkyl or C₁ to C₆ substituted alkyl.

2. The composition of claim 1, wherein the metal ion is selected from the group consisting of calcium, chromium, cobalt, copper, iron, magnesium, manganese, nickel, potassium, sodium, and zinc.

3. The composition of claim 2, wherein the ratio of the ligand to the metal ion is from about 1:1 to about 3:1.

4. The composition of claim 1, wherein the phytase is of fungal, yeast, or bacterial origin.

5. The composition of claim 1, wherein R¹ is methyl and n is 2.

6. The composition of claim 5, wherein the metal ion is selected from the group consisting of zinc, copper, and manganese.

7. The composition of claim 6, wherein the ratio of the ligand to the metal ion is about 2:1.

8. A method for improving dietary phosphorus and calcium utilization in an animal, the method comprising administering to the animal a metal chelate and a phytase, the metal chelate having Formula (I):
LₓM^{y} (I)
wherein:
L is a ligand;
M is a metal ion; and
x and y are integers from 1 to 10; and
wherein dietary phosphorus and calcium utilization are increased relative to administration of an inorganic salt of the metal ion and the phytase.

9. The method of claim 8, wherein L is an organic acid moiety selected from the group consisting of adipate, ascorbate, caprylate, citrate, furmarate, glucoheptonate, gluconate, glutarate, glycerophosphate, lactate, ketoglutarate, malate, malonate, orotate, oxlate, pantothenate, picolinate, pidolate, sebacate, succinate, and tartrate; or L is an amino acid moiety selected from the group consisting of alanate, arginate, asparaginate, aspartate, cysteinate, glutaminate, glutamate, histidinate, homocysteinate, isoleucinate, lysinate, methionate, phenylalinate, prolinate, serinate, threonate, typtophanate, tyrosinate, and valinate.

10. The method of claim 8, wherein Lisa compound of Formula (III): 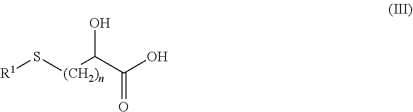
wherein
n is an integer from 1 to 5; and
R¹ is C₁ to C₆ alkyl or C₁ to C₆ substituted alky.

11. The method of claim 8, wherein the metal ion is selected from the group consisting of calcium, chromium, cobalt, copper, iron, magnesium, manganese, nickel, potassium, sodium, and zinc.

12. The method of claim 11, wherein.

13. The method of claim 10, wherein R¹ is methyl, and n is 2.

14. The method of claim 13, wherein the metal ion is selected from the group consisting of zinc, copper, and manganese.

15. The method of claim 14, wherein each of x and y is 2.

16. The method of claim 8, wherein the phytase is of fungal, yeast, or bacterial origin.

17. The method of claim 8, wherein the amount of the metal ion administered to the animal as the metal chelate is from about 10 to about 200 ppm; and the amount of the phytase administered to the animal is from about 100 to about 1000 phytase units (FTU) per kg.

18. A method for improving dietary phosphorus and calcium utilization in an animal fed a diet comprising a phytase, the method comprising administering to the animal a metal chelate having Formula (I):
LₓM^{y} (I)
wherein:
L is a ligand;
M is a metal ion; and
X and y are integers from 1 to 10; and
wherein dietary phosphorus and calcium utilization are increased relative to administration of an inorganic salt of the metal ion.

19. The method of claim 18, wherein L is an organic acid moiety selected from the group consisting of adipate, ascorbate, caprylate, citrate, furmarate, glucoheptonate, gluconate, glutarate, glycerophosphate, lactate, ketoglutarate, malate, malonate, orotate, oxlate, pantothenate, picolinate, pidolate, sebacate, succinate, and tartrate; or L is an amino acid moiety selected from the group consisting of alanate, arginate, asparaginate, aspartate, cysteinate, glutaminate, glutamate, histidinate, homocysteinate, isoleucinate, lysinate, methionate, phenylalinate, prolinate, serinate, threonate, typtophanate, tyrosinate, and valinate.

20. The method of claim 18, wherein Lisa compound of Formula (III): 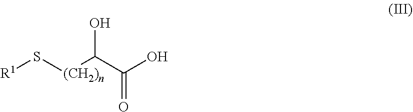
wherein
n is an integer from 1 to 5; and
R¹ is C₁ to C₆ alkyl or C₁ to C₆ substituted alky.

21. The method of claim 18, wherein the metal ion is selected from the group consisting of calcium, chromium, cobalt, copper, iron, magnesium, manganese, nickel, potassium, sodium, and zinc.

22. The method of claim 21, wherein each of x and y is from 1 to 3.

23. The method of claim 20, wherein R¹ is methyl, and n is 2.

24. The method of claim 23, wherein the metal ion is selected from the group consisting of zinc, copper, and manganese.

25. The method of claim 24, wherein each of x and y is 2.

26. The method of claim 18, wherein the amount of the metal ion administered to the animal as the metal chelate is from about 10 to about 200 ppm.
